SEQUENCE LISTING (2 sequences)

SEQ ID NO: 1 - protein, 17 aa, Homo sapiens
CYYITGNLETFPRDPEK

SEQ ID NO: 2 - protein, 16 aa, Artificial Sequence
  control peptide
CTKTPLDQHTLQGDQA

(19) United States
(12) Patent Application Publication
(10) Pub. No.: US 2007/0054378 A1
(43) Pub. Date: Mar. 8, 2007

(54) Elicitation of antibodies to self peptides in mice by immunization with dendritic cells

(75) Inventors: Katherine S. Bowdish, Del Mar, CA (US); Anke Kretz-Rommel, San Diego, CA (US)

Correspondence Address: FISH & NEAVE IP GROUP;ROPES & GRAY LLP, ONE INTERNATIONAL PLACE, BOSTON, MA, 02110-2624, US

(73) Assignee: Alexion Pharmaceuticals, Inc., Cheshire, CT (US)

(21) Appl. No.: 10/572,132

(22) PCT Filed: Sep. 18, 2004
(86) PCT No.: PCT/US04/30556

§ 371 Date: Aug. 11, 2006

Related U.S. Application Data
(60) Provisional application No. 60/503,738, filed on Sep. 18, 2003.

(51) Int. Cl. C12P 21/04; C12N 5/06

(52) U.S. Cl. 435/70.21; 435/326

(57) ABSTRACT

Antibodies are produced by inoculating a host animal with dendritic cells in combination with a self peptide of interest. In one embodiment, antibodies are produced by grafting tissue from one or more human immune organs into an immunodeficient, non-human, mammalian host such as, for example, a SCID mouse and then immunizing the host with a self peptide of interest.

### RELATED APPLICATIONS

This application claims priority to U.S. Provisional Application Ser. No. 60/503,738, filed Sep. 18, 2003, the contents of which are incorporated herein in their entirety.

### BACKGROUND

1. Technical Field

The present disclosure relates to methods for rapidly raising antibodies to self peptides using dendritic cells.

2. Background of Related Art

The major histocompatibility complex is a series of genes that code for protein molecules responsible for cell-cell recognition and interaction. The MHC of mammalian species contains three groups of genes: class I, class II, and class III. Class I and class II genes code for cell surface recognition molecules. Class III genes code for certain complement components.

The ability of cells to recognize other cells as self or as originating from another genetically different individual (non-self) is an important property in maintaining the integrity of tissue and organ structure. Class I and class II MHC products control recognition of self and non-self. The major histocompatibility system thus prevents an individual from being invaded by cells from another individual. For example, transplants from one individual generally cannot survive in another individual because of histocompatibility differences.

Histocompatibility similarities are required for cellular cooperation in induction of the immune response, and they provide a mechanism to ensure that T cells and B cells of a given individual can recognize each other for cooperation, yet recognize foreign structures at the same time. In general, T lymphocytes recognize antigens and respond to them when they are presented on the surface of an antigen-presenting cell (APC).

Dendritic cells are one of the most potent groups of antigen-presenting cells. Dendritic cells (DCs) are highly specialized APCs, capable of activating naive and memory T lymphocytes. Dendritic cells are derived from bone marrow progenitor cells and monocytes. Immature dendritic cells (iDCs) are found under the skin and mucous membranes where they engulf microorganisms and antigenic molecules through phagocytosis and pinocytosis. Subsequently, the immature dendritic cells migrate to the follicles of secondary lymphoid organs such as lymph nodules, lymph nodes and the spleen and, in the process, mature into mature dendritic cells (mDCs), a class of professional APCs. Mature dendritic cells have numerous pseudopodia-like projections to increase their surface for antigen presentation. DCs degrade foreign microorganisms and other materials with their lysosomes.

DCs are unique in their ability to interact with and activate resting T cells. They are uniquely capable of sensitizing naive T cells to protein antigens and eliciting antigen specific immune responses. Peptides from microbial proteins are bound to MHC-II molecules, which are produced by macrophages, dendritic cells, and B-lymphocytes. The peptide epitopes bound to the MHC-II molecules are then placed on the surface of the dendritic cell where they can be presented to T4-lymphocytes. In addition, dendritic cells can bind peptide epitopes to MHC-I molecules and present them to T8-lymphocytes. Dendritic cells also use toll-like receptors to recognize pathogen-associated molecular patterns. This interaction stimulates the production of co-stimulatory molecules that are also required for T-lymphocyte activation.

Naive T cells are characterized by a high expression of ICAM-3 which is a member of the IgG supergene family and is rapidly downregulated after activation (Vazeux et al., 1992). Studies of murine and human dendritic cells isolated from peripheral blood indicate that these cells can be used to stimulate and expand antigen specific CD4+ and CD8+ cells in vitro. Shen et al., “Cloned Dendritic Cells can Present Exogenous Antigens on Both MHC Class I and Class II Molecules,” J. Immunol. 158: 2723 (1997). Cancer vaccines have also been developed loading dendritic cells in vitro with tumor lysate, tumor proteins and/or tumor peptides. See, e.g., Ashely, et al., “Bone Marrow-Generated Dendritic Cells Pulsed with Tumor Extracts or Tumor RNA Induce Antitumor Immunity Against Central Nervous System Tumors,” J. Exp. Med. 186: 1177 (1997); Avigan, “Dendritic Cells: Development, Function and Potential Use for Cancer Immunotherapy,” Blood Rev. 13: 51 (1999); Zhou, et al., “Current Methods for Loading Dendritic cells with Tumor Antigen for the Induction of Antitumor Immunity,” J. Immunother. 25: 289 (2002). However, tumor antigens can be recognized by the body as foreign since cytotoxic T cells and antibodies against tumor antigens can be found in cancer patients. Platsoucas, et al., “Immune Responses to Human Tumors: Development of Tumor Vaccines,” Anticancer Res. 23: 1969 (2003).

A number of adhesive interactions between the DC and T lymphocyte are important during T lymphocyte activation. The DC-expressed beta-integrin, LFA-1, was originally described as the major ligand for intercellular adhesion molecule (ICAM-3) on T-lymphocytes. However, antibodies against LFA-1 failed to abrogate T-cell-DC clustering, while anti-ICAM-3 completely abrogated this clustering. A search thus began for the dendritic cell expressed molecule responsible for this ICAM-3-binding activity.

Recently, a novel ICAM-3 binding C-type lectin, known as DC-Specific ICAM-3 grabbing non-integrin, or DC-SIGN, was found. DC-SIGN is expressed on dendritic cells and appears to mediate adhesion between dendritic cells and ICAM-3 on naïve T cells and to be essential for DC-induced T cell proliferation (See Geijtenbeek et al., Cell 100: 587-597 (2000)).

WO 00/63251, the contents of which are incorporated by reference herein, describes the use of DC-SIGN to modulate immune responses by affecting the interaction between dendritic cells and T cells. Immune responses can be inhibited or prevented by preventing the interaction of DC-SIGN on dendritic cells with receptors on T cells, e.g., by using antibodies specific for DC-SIGN. Alternatively, an immune response to an antigen can be potentiated by binding the antigen to DC-SIGN on dendritic cells such that the antigen plus DC-SIGN is taken up by dendritic cells and processed and presented to T cells.

Cytokines also play a role in directing the T cell response. Helper (CD4+) T cells orchestrate the immune response of mammals through production of soluble factors that act on other immune system cells, including other T cells. Most mature CD4+T helper cells express one of two cytokine profiles: Th1 or Th2. Th1 cells express IL-3, IL-4, IL-5, IL-6, IL-9, IL-10, IL-13, GM-CSF and low levels of TNF-A. The TH1 subset promotes delayed-type hypersensitivity, cell-mediated immunity, and immunoglobulin class switching to IgG₂ₐ. The Th2 subset induces humoral immunity by activating B cells, promoting antibody production, and inducing class switching to IgG₁ and IgE. Dendritic cells produce cytokines such as tumor necrosis factor-alpha (TNF-α), interleukin-1 (Il-1), interleukin-6 (Il-6), and interleukin-8 (Il-8).

A number of bacterial products, such as lipopolysaccharide, are also known to stimulate mammalian immune responses. Recently, bacterial DNA itself has been reported to be one such molecule (e.g., Krieg, A. M., et al., 1995, Nature 374:546-9). One of the major differences between bacterial DNA, which has potent immunostimulatory effects, and vertebrate DNA, which does not, is that bacterial DNA contains a higher frequency of unmethylated CpG dinucleotides than does vertebrate DNA. Select synthetic oligodeoxynucleotides (ODN) containing unmethylated CpG motifs (CpG ODN) have been shown to have an immunologic effects and can induce activation of B cells, NK cells and APCs such as monocytes and macrophages (Krieg, A. M., et al., supra). They can also enhance production of cytokines known to participate in the development of an active immune response, including tumor necrosis factor-α, IL-12 and IL-6 (e.g., Klinman D. M., et al., Proc. Natl. Acad. Sci. USA, 93:2879-83, 1996).

Phage libraries displaying antibody fragments are very useful for the discovery of specific antibodies directed against human proteins. It is highly desirable to identify antibodies from a library derived from an immunized individual. Since it is unethical to immunize humans except for approved vaccines, other mammals are used to produce such libraries. Frequently, mice are the species of choice in producing such libraries. In certain applications, antibodies may be raised against specific peptides. In general, this is accomplished by linking peptides to carrier proteins such as KLH or BSA since an immune response against the peptide alone cannot be achieved. However, one disadvantage to this approach is that the immune response will be raised primarily against the carrier protein. In addition, human peptides or proteins are frequently homologous to mouse proteins, thus immunizations with self proteins are typically unsuccessful, even in the presence of carrier proteins.

Improved methods for raising antibodies to peptides, especially self peptides, are thus desirable.

### SUMMARY

The present disclosure is directed to methods for producing antibodies to self polypeptides. In one embodiment, the method comprises the steps of isolating dendritic cells from a host animal; incubating the dendritic cells with a self polypeptide of interest to form an immunogen; immunizing the host animal with the immunogen; and obtaining antibodies to the self polypeptide of interest.

In another embodiment, human antibodies are produced using non-human hosts. In this embodiment, antibodies are produced by grafting human tissue into an immunodeficient, non-human, mammalian host animal, the human tissue comprising at least a sufficient portion of thymus, spleen, bone marrow, skin, lymph nodes and fetal liver to produce a human antibody; applying a composition to the grafted human tissue, the composition including at least one growth factor and a material to support cell growth; obtaining human monocytes from a donor; maturing the human monocytes to dendritic cells; isolating the dendritic cells; incubating the dendritic cells with a self polypeptide of interest to form an immunogen; immunizing the host with the immunogen; and harvesting at least a portion of the grafted tissue that includes cells that produce human antibodies to the self polypeptide of interest.

In yet another embodiment, methods of producing human antibodies in vivo in non-human hosts are provided. This method includes grafting human tissue into an immunodeficient, non-human, mammalian host animal, the human tissue comprising at least a sufficient portion of thymus, spleen, bone marrow, skin, lymph nodes and fetal liver to produce a human antibody; applying a composition to the grafted human tissue, the composition including at least one growth factor and a material to support cell growth; linking a self peptide of interest to an antibody to DC-Specific ICAM-3 grabbing non-integrin; immunizing the host with the self polypeptide of interest and antibody to DC-Specific ICAM-3 grabbing non-integrin; and harvesting at least a portion of the grafted tissue that includes cells that produce human antibodies to the self polypeptide of interest.

Antibodies produced by the methods of the present disclosure are also provided.

The present disclosure also provides a method of testing the effect of drugs comprising administering a drug to a non-human mammalian having at least a sufficient portion of human thymus, spleen, bone marrow, skin, lymph nodes and fetal liver grafted therein to allow the development of one or more types of human immune cells selected from the group consisting of T cells, B cells and dendritic cells; and observing any effects of the drug on human antibody production.

### BRIEF DESCRIPTION OF THE DRAWINGS

FIG. 1 is a graphical depiction of ELISA results demonstrating the binding of antibodies produced in accordance with the present disclosure to a self peptide of interest.

FIG. 2 is another graphical depiction of ELISA results demonstrating the binding of antibodies produced in accordance with the present disclosure to a self peptide of interest.

### DETAILED DESCRIPTION OF PREFERRED EMBODIMENTS

The present disclosure is directed to methods for rapidly raising antibodies directed against self polypeptides using dendritic cells. Preferably, a response is elicited to a self polypeptide localized on the cell surface of a dendritic cell. Phage libraries displaying antibodies or antibody fragments to these polypeptides may then be constructed and utilized to identify additional antibodies directed against human or mouse polypeptides.

The term “self polypeptide” as used herein means any polyamino acid (including, but not limited to peptides, full length proteins and truncated proteins) produced by a normal, healthy subject that does not elicit an immune response within the subject. It should be understood that the self polypeptides can be naturally occurring or synthetically produced. It also should be understood that the self polypeptide need not be identical to the naturally produced polypeptide, but rather can include variations thereto having homology as low as 60%, provided the variation does not elicit an immune response within the subject.

In accordance with the present disclosure, a suitable mammalian host for immunization is first identified. Preferably the host animal is a rodent, such as a rat, a hamster or a mouse. Most preferably a Swiss, Balb/c or NIH mouse is utilized as the host animal.

The dendritic cells may be obtained from bone marrow, blood, the spleen, or any other source. In one embodiment, the dendritic cells are obtained from the spleen of the host animal.

Methods for obtaining dendritic cells from a host are known to those skilled in the art. For example, where the source of the dendritic cells is the spleen, cells may be obtained by digesting the spleen with an enzyme such as collagenase, manually teasing the spleen apart, and then filtering the cells through a filter. Dendritic cells may be selected using methods known to those skilled in the art, and include the use of systems utilizing antibodies to markers associated with DCs. Such markers include, but are not limited to, cell surface molecules responsible for T cell activation such as class I and class II MHC, adhesion molecules (e.g., CD54 and CD11), and costimulatory molecules (e.g., CD40, CD80, CD86 and 4-1BB). Commercially available systems which may be utilized include Stemcell Technologies' EasySep system, which involves labeling dendritic cells with anti-CD11c coated magnetic beads.

Once obtained, purified dendritic cells may be incubated in an appropriate incubator, such as a CO₂ incubator, for a period of time ranging from about 30 minutes to about 3 hours, more preferably from about 45 minutes to about 90 minutes with a polypeptide of interest. As used herein, a polypeptide of interest can be both haptens and antigens, where the haptens are modified to provide for an immune response. Most preferably, the peptide of interest is a self polypeptide. Preferably, the polypeptide of interest obtained from the host animal is homologous with a human polypeptide. The degree of homology between the polypeptide in the host animal and human can vary, preferably ranging from about 60% to about 100% homology, more preferably from about 90% to about 100% homology, with 100% homology between the human and host animal polypeptide being most preferred.

It is further contemplated that the polypeptide of interest can be immobilized on a substrate prior to contact with the dendritic cells. Any suitable substrate within the purview of those skilled in the art can be used to carry the polypeptide of interest. Typically, the substrate will be an inert synthetic materials. In one embodiment, polystyrene beads are used ad the substrate and are coated with the polypeptide of interest. The coated beads are then incubated with dendritic cells.

The dendritic cells and polypeptide of interest (alone or coated onto a substrate) are placed in an appropriate medium for the growth of such cells including, but not limited to, Eagle's Minimal Essential Medium with Earle's salts (EMEM) (GIBCO, Grand Island, N.Y.). Where the source of the dendritic cells is murine, the incubation preferably occurs in media containing from about 1% to about 10% mouse serum, with a range of from about 2% to about 5% mouse serum preferred. The combination of dendritic cells and peptide of interest is sometimes referred to herein as an “immunogen”.

Alternatively, the dendritic cells are transfected with nucleic acid encoding a polypeptide of interest (e.g., with RNA encoding a polypeptide of interest), and then cultured to activate the dendritic cells. Techniques for tranfecting a dendritic cell with nucleic acid encoding a polypeptide of interest are within the purview of those skilled in the art.

In addition to loading dendritic cells with a polypeptide of interest, B cells can also optionally be incubated with the polypeptide of interest. Techniques for incubating B cells with a polypeptide of interest are within the purview of those skilled in the art. Both cell types (the dendritic cells and B cells) that have been contacted with the polypeptide of interest can be injected into a host animal at the same time.

The immunogen (alone or optionally together with B cells that have been incubated with a polypeptide of interest) can then be introduced into a host animal. The entire culture medium, (including unbound polypeptide, dendritic cells, culture medium, immunogen, etc.), can be administered to the host animal. Alternatively, the cells can be washed and, in some embodiments, an adjuvant added prior to introduction into a host animal. Suitable adjuvants include cytokines and similar compounds which help orchestrate an immune response to the peptide of interest on the dendritic cells. As used herein, the term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to IL-1, IL-2, IL4, IL-5, IL-6, IL-7, IL-10, IL-12, IL-15, granulocyte-macrophage colony stimulating factor (GM-CSF), granulocyte colony stimulating factor (GCSF), interferon-γ (γ-INF), tumor necrosis factor (TNF), TNF-α, TGF-β, Flt-3 ligand, and CD40 ligand. CpG oligonucleotide, which enhances antibody dependent cellular cytotoxicity, can also be used as an adjuvant in conjunction with peptide/antigen presentation. Other adjuvants include alum, RIBI, complete Freund's adjuvant, specol, B. pertussis or its toxin, etc.

Preferably, the adjuvant is a CpG oligonucleotide (commercially available from Qiagen (Chatsworth, Calif.)) or Flt-3 (U.S. Pat. No. 6,190,655). The combination of adjuvant and polypeptide of interest enhances the production of an immune response against the polypeptide of interest.

Serum is obtained from the host prior to introduction of the immunogen and adjuvant, if any. The immunogen in combination with adjuvant, if any, may then be administered to the host. Administration will normally be by injection, either subcutaneous, intramuscular, intraperitoneal or intravascular. It should be understood that multiple injections can be performed over a period of time. Where multiple injections are used, subsequent injections may be made, within 1 to 6, more usually 2 to 4 weeks of the previous injection. In subsequent injections, dendritic cells from the same subject, or a related subject can be used, either alone or conjugated to the polypeptide of interest. In addition, subsequent injections may include the same or different adjuvants.

After a time sufficient for the formation of antibodies, which can range from about 7 to about 21 days, preferably from about 7 to about 10 days from the last injection, a blood sample is taken and the sample is subjected to an assay for antibodies to the polypeptide of interest.

Various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention

In an alternate embodiment, human antibodies are produced by human tissue grafted into a non-human, mammalian recipient. Thus, in addition to raising mouse antibodies, it is also possible to raise human antibodies in mice grafted with components of the human immune system. The grafted tissue includes sufficient portions of one or more human immune organs to produce a fully human antibody. The term “human immune organ” is intended to include human tissue from any organ that produces one or more molecules involved in the production of a human antibody. Thus, for example, tissue containing or capable of developing cell types that actually produce antibodies are included in the graft. In addition, tissue involved in the recognition, breakdown or presentation of an antigen to an antibody producing cell may also be included in the grafted tissue. Suitable tissue types which fall within the category of “human immune organ” include of thymus, spleen, bone marrow, skin, lymph nodes and liver. Other suitable types of tissue will be readily apparent to those skilled in the art.

The tissue to be grafted is preferably obtained from a fetal source, having a gestational age of at least four months, more usually in the range of 18 to 24 weeks and ranging up to neonate tissue, depending upon the nature of the tissue or organ. Tissue from a single donor is preferred. The use of fetal instead of adult tissues is preferred because it allows development of the immune system directly within the recipient. As the fetal cells develop they are exposed to proteins native to the recipient, and the immune organs become tolerant to the environment within the recipient.

In a particularly useful embodiment, thymus, spleen, bone marrow, skin, lymph nodes and fetal liver are all grafted into the recipient to ensure that all aspects of antibody production are provided. Transplanting bone, fetal liver and thymus allows the development of T and B cells, thereby providing a broad repertoire of potential antigen specificity. Addition of skin provides the site of immunogen injection (as described in more detail below) with the highest probability of human antigen presenting cells picking up the immunogen. Lymph nodes can be important for T/B cell interaction and the spleen is a potential resource of B cells to construct a display library.

The amount of tissue grafted into the recipient should be an amount sufficient to produce antibodies. The amount of tissue grafted can be as little as 1 mm³ of each type of tissue. On the other hand, grafts of 100 mm³ or more can be used, depending on the specific recipient chosen.

The tissue may be fresh tissue, obtained within about 48 hours of death, or cryopreserved in a manner that maintains viability of the tissue.

The recipient can be any type of animal into which human tissue can be grafted and remain viable. Genetically immunocompromised mice are a particularly preferred recipient, especially NOD-SCID mice. In such a case, a mouse having such human tissue grafted therein is sometimes referred to as a “humouse”.

Where possible, it is preferred to irradiate the recipient before the human tissue is grafted therein. As those skilled in the art will appreciate, irradiation of the recipient will eliminate the native immune function of the recipient. The specific parameters for irradiation will depend on the particular type of recipient, especially the volume of the recipient. It should be understood, of course, that the conditions for irradiation should not be sufficiently severe as to kill the recipient. Irradiation conditions for various types of recipients are known to those skilled in the art. In general, the lowest dose of radiation necessary to cause loss of immune function should be applied. By starting low and applying progressively higher doses of radiation, an effective dosage of radiation can be determined without undue experimentation. SCID mice can be irradiated with gamma radiation in an amount ranging from 100 to 2000 RAD.

Grafting can be accomplished by simply making an incision in the skin of the recipient and placing the human tissue under the skin of the recipient. If desired, the grafted tissue can be secured to a desired location within the recipient's body such as, for example by sutures or staples. The location at which the human tissue is grafted is not critical. Considerations in choosing a location for the graft include the likelihood that the grafted tissue will be vascularized, the ease of implantation and the ease of retrieval of the implanted tissue. Preferably the grafting site is a highly vascularized location. The site of grafting can be downstream from a convenient site in the blood or lymphatic system for introduction of an antigen as described below. In particularly useful embodiments, primary lymph organs (e.g., bone, liver and thymus) are grafted at a location remote from the secondary lymph organs (e.g., spleen, lymph nodes and skin). Once the tissue is placed and/or secured within the recipient, the incision in the recipient's skin can be closed (e.g., by suturing, stapling or adhesive). While the human skin can be placed below the recipient's skin, it is preferred to remove a section of the recipient's skin and provide a surface graft of the human skin to allow injection of an immunogen directly through the human skin graft. Optionally, the mononuclear fraction of cord blood can be injected into the recipient's blood stream at or around the time of grafting.

At or after the placement of the graft, the immediate environment surrounding the graft is optionally treated to enhance lymphocyte development and antibody production. Suitable treatments include the application of compositions containing components known to enhance vascularization and cell growth, the differentiation of cells and/or the production of antibodies. Suitable compositions include gelling agents (such as, for example, methylcellulose or agar) containing growth factors (such as, for example, Stem Cell Factor, Granulocyte Macrophage-Stimulating Factor, IL-3, IL-6, Granulocyte-Colony Stimulating Factor, and erythropoietin). Commercially available products that can be used for this purpose include METHOCULT® GF H4435 available from Stemcell Technologies Inc., Vancouver, British Columbia. Details regarding METHOCULT® products are disclosed in the following articles: Conneally E, Bardy P, Eaves C J, Thomas T, Chappel S, Shpall E J, Humphries R K: Rapid and efficient selection of human hematopoietic cells expressing murine heat-stable antigen as an indicator of retroviral-mediated gene transfer. Blood 87: 456, 1996; Eaves C J: Assays of hemopoietic progenitor cells. Williams Hematology, 5 (eds. E Beutler, M A Lichtman, B S Coller, T J Kipps), McGraw-Hill, Inc., pp L22-6, 1995; Eaves C, Lambie K: Atlas of Human Hematopoietic Colonies, StemCell Technologies Inc, 1995; Eaves A C, Eaves C J: Current Therapy in Hematology-Oncology 4: 159, 1992; Helgason C D, Sauvageau G, Lawrence H J, Largman C, Humphries R K: Overexpression of HOXB4 enhances the hematopoietic potential of embryonic stem cells differentiated in vitro. Blood 87: 2740, 1996; Hogge D E, Lansdorp P M, Reid D, Gerhard B, Eaves C J: Enhanced detection, maintenance and differentiation of primitive human hematopoietic cells in cultures containing murine fibroblasts engineered to produce human Steel factor, interleukin-3 and granulocyte colony-stimulating factor. Blood 88: 3765, 1996; Hough M R, Chappel M S, Sauvageau G, Takei F. Kay R, Humphries R K: Reduction of early B lymphocyte precursors in transgenic mice overexpressing the murine heat-stable antigen. J Immunol 156: 479, 1996; Lemieux M E, Rebel V I, Lansdorp P M, Eaves C J: Characterization and purification of a primitive hematopoietic cell type in adult mouse marrow capable of lympho-myeloid differentiation in long-term marrow “switch” cultures. Blood 86: 1339, 1995; Mayani H, Dragowska W, Lansdorp P M: Cytokine-induced selective expansion and maturation of erythroid versus myeloid progenitors from purified cord blood precursor cells. Blood 81: 3252, 1993; Petzer A L, Hogge D E, Lansdorp P M, Reid D S, Eaves C J: Self-renewal of primitive human hematopoietic cells (long-term culture-initiating cells) in vitro and their expansion in defined medium. Proc Natl Acad Sci USA 93: 1470, 1996. The disclosure of each of these references is incorporated by reference herein.

Another useful composition that can be applied to the grafted human tissue is a composition capable of supporting cell growth. Suitable compositions in this category include basement-membrane-derived compositions containing a biologically active polymerizable extract containing laminin, collagen IV, nidogen, heparin sulfate proteoglycan and entactin. The term “biologically active” as used in connection with this basement-membrane-derived composition means capable of supporting normal growth and differentiation of various cell types when cultured including epithelial cells. One such composition is commercially available under the tradename MATRIGEL® Basement Membrane Matrix available from Becton Dickinson Labware, Bedford Mass. Details regarding MATRIGEL® products are disclosed in U.S. Pat. No. 4,829,000, the disclosure of which is incorporated herein by reference. A combination of such materials can advantageously be employed.

The composition(s) can be applied directly to the location of grafted organ at the time of grafting and/or subsequent to grafting by injection to the site of grafting. The amount of the composition applied is not critical and will depend on such factors as the type(s) and amount of human tissue grafted, the specific composition employed, the specific recipient, and the location of the graft. Typically from about 100 μl to about 500 μl of the composition will be applied at the time of grafting. In particularly useful embodiments, the composition is re-applied after grafting at intervals of between seven and ten days until immunization, as discussed in more detail below.

After the grafting is accomplished, the recipient is monitored to ascertain whether human antibodies are being produced. Techniques for detection of human antibodies are within the purview of one skilled in the art. Kits for detecting the presence of human antibodies in an animal's serum such as, for example, Easy-Titer® human IgG assay kit are commercially available from Pierce (Rockford, Ill.). Normally, by the point in time when human antibodies are detected, the grafted tissue remains viable, has vascularized and is believed to have lymphatic vessels connected thereto. Generally, at least one week will transpire, however anywhere from 2 to 20 weeks or more may be allowed to pass before the next step (immunization) is performed.

As alternative to the NOD/SCID mouse system described above, the use RAG/γc −/− mice that have been injected with CD34+ human cord blood cells as described by Traggiai et al. (see, Science. Apr. 2, 2004;304(5667):104-7, the disclosure of which is incorporated herein by this reference) is contemplated. As noted by Traggiai et al., intrahepatic injection of CD34+ human cord blood cells into conditioned newborn Rag2−/−γc−/− mice can lead to de novo development of B, T, and dendritic cells; formation of structured primary and secondary lymphoid organs; and production of functional immune responses. Some cordblood cells can be matured in vitro with GM-CSF and IL-4 to immature dendritic cells. At that point, cells can be either frozen or protein can be added and the cells matured with a cocktail of TNF-α, IL-6, PGE2 or similar dendritic cell maturation cocktails into mature dendritic cells and then injected into the mice. The frozen immature dendritic cells can be used at a later time. The frozen cells can be defrosted and matured in the presence of protein as described above and then injected into the mice between 2-20 weeks of age.

Once it is detected that human antibodies are present in the recipient, the recipient is immunized with dendritic cells in combination with a polypeptide of interest, preferably a self polypeptide. Because of the grafting of human tissue, the self polypeptide of interest may be human. Preferably, dendritic cells are matured from monocytes obtained from a suitable human donor; most preferably the donor of the human tissue utilized in the graft or a human leucocyte antigen (HLA)-matched human donor. The monocytes are then allowed to mature to dendritic cells. Dendritic cells are isolated and incubated with a polypeptide of interest under conditions as described above to produce an immunogen. The immunogen may also be combined with a wide variety of adjuvants as described above.

In a preferred embodiment, an immunizing composition is prepared by culturing the mononuclear fraction of cord blood to grow dendritic cells. The dendritic cells are then stimulated with the self polypeptide. The resulting immunogen is then injected into the recipient to accomplish immunization.

The immunogen can be administered systemically to the recipient of the graft or injected locally at the site of the graft. Administration will normally be by injection, either subcutaneous or intravascular, preferably directly into the site of the grafted human tissue. In particularly useful embodiments, the recipient has received a human skin graft and the immunogen is injected through the grafted skin or adjacent to it. Considerations in determining how much immunogen should be administered include the nature of the immunogen, the amount of tissue grafted into the recipient and the desired degree and swiftness of the immune response sought. One or more booster injections maybe made, usually within 1 to 6, more usually 2 to 4 weeks of the previous injection. The booster injection may have the same composition or different composition than the prior injection. Immunization may also be accomplished by the RIMMS technique which is well known to those skilled in the art.

The recipient is then monitored to ascertain whether an immunogen-specific response has been mounted. Techniques for detection of antibodies specific to any given immunogen are within the purview of one skilled in the art. One suitable method of detecting the presence of human immunogen-specific antibodies in an animal's serum is disclosed in Current Protocols in Immunology, Coligan et al., Chapter 2.1 (John Wiley & Sons, 2000 ed.), the disclosure of which is incorporated herein by reference.

Once the immunogen-specific immune response is detected, at least a portion of the grafted tissue is removed from the recipient. Mouse tissue into which human cells may have migrated may also be extracted to maximize the amount of antibody producing cells collected.

In yet another embodiment, antibody response may be elicited by targeting dendritic cells in vivo. It has been recently shown that antibodies directed against certain surface receptors on dendritic cells such as DC-SIGN are internalized and presented to T cells. See Engering, et al., “The dendritic cell-specific adhesion receptor DC-SIGN internalizes antigen for presentation to T cells,” J. Immunol. 168: 2118 (2002). Antibodies to DC-SIGN may be constructed as disclosed in WO/0063251 and linked to a peptide of interest using techniques known to those skilled in the art. The antibody and peptide of interest may then be injected into a mouse, preferably a humouse, to induce a strong antibody response against the peptide of interest. Antibodies are then obtained using techniques known to those skilled in the art as described above.

Once obtained, cells or genetic material from the host can be immortalized and/or cloned as desired. Thus, for example, RNA from the removed tissue is isolated using techniques well known to those skilled in the art. The recovered RNA is used to generate one or more antibody libraries and the libraries are screened to identify antibodies that bind to the peptide of interest or a component thereof. In preferred embodiments, the screening process, also known as panning, is conducted to identify antibodies that have either an agonistic or antagonistic effect on the peptide of interest. Alternatively, cells of interest can be separated by fluorescence activated cell sorter (FACS) sorting or magnetic sorting. Techniques for producing and screening antibody libraries are well known to those skilled in the art. See, for example, U.S. Pat. No. 6,291,161 to Lerner et al., and U.S. patent application Ser. Nos. 10/014,012 and 10/251,085, the disclosures of each of which are incorporated herein in their entirety.

In one embodiment, phage expressing antibody fragments on their surface can be produced and concentrated so that all members of a library can be allowed to bind to the peptide of interest. The peptide of interest can be immobilized on a microtiter dish, on whole cells, the membranes of whole cells, or present in solution. Non-specific Ab-phage are washed away, and bound phage particles are released from the peptide of interest, often by the use of low pH. The recovered Ab-phage are infectious and so can be amplified in a bacterial host. Typically, multiple rounds of this sort of selection are performed. Individual antibody fragment clones can then be analyzed as soluble Fabs or scFvs for identification of those that specifically recognize the peptide of interest. Additional methods for the construction of such phage display libraries, including those displaying Fab, and methods for panning are disclosed in U.S. patent application Ser. Nos. 09/235,499 and 10/006593, the contents of each of which are incorporated by reference herein.

As another example, antibody-producing cells can be selected and fused with non-antibody producing cells such as, for example, immortalized cell lines. These fusion partners are typically transformed human cells such as human myeloma cells. One suitable human myeloma cell line is disclosed by Karpas, et al., PNAS, vol. 94, no. 4, pages 1799-1804, 2001. After fusion, fused cells are segregated into individual cultures and propagated, and hybridoma lines which express immunogen-specific monoclonal antibodies are selected. These cell lines can be maintained in culture or cryopreserved using techniques well known to those of ordinary skill in the art.

The hybridomas may then be introduced into host animals, e.g. mice or rats, to produce ascites fluid or mechanically expanded, using spinner flasks, roller bottles, etc, The host will be immunocompromised, so as to be able to accept the neoplastic graft.

Antibodies produced in accordance with the present disclosure may be used in a variety of ways, both diagnostic and therapeutic. For example, antibodies to the self polypeptide produced by the KIAA0746 gene can be produced in accordance with the present disclosure. These self polypeptides are upregulated in patients suffering from colon cancer. Thus, antibodies to these self polypeptides can be used to diagnose colon cancer. Alternatively, the antibody can be used a targeting agent and can be conjugated with therapeutic agents (such as those known to be useful for treating colon cancer) and administered to colon cancer patients as therapy.

In one embodiment, the subject antibodies may be used in the treatment of disease, neutralizing viruses or other pathogens, for in vivo diagnoses, for targeted toxicity against neoplastic cells or precursors to such cells, passive immunization, in conjunction with transplantation, and the like. The subject antibodies may be modified by radiolabeling, conjugation to other compounds, such as biotin, avidin, enzymes, cytotoxic agents, e.g. ricin, diphtheria toxin, arbin, etc., and the like.

Where the antibodies are produced in a humouse, the non-human recipients of the grafted human immune organ can be used in methods for testing of the effects of drugs on the human immune system. In these methods, a drug is administered to the non-human mammal host having at least a sufficient portion of thymus, spleen, bone marrow, skin, lymph nodes and fetal liver grafted therein to produce a human antibody. After a period of time, any effects of the drug on human antibody production is observed. Techniques for determining the effects of a drug on a human immune system are known to and within the purview of those skilled in the art.

The following non-limiting examples are provided to illustrate the methods described herein.

### EXAMPLE 1

Dendritic cells from mouse spleen were isolated by first digesting the spleen with 1 mg/ml collagenase for 1 hour, teasing the spleen apart and filtering the cells through a mesh 40 filter. Dendritic cells were positively selected using Stemcell Technologies' EasySep system labeling the cells with anti-CD11c coated magnetic beads. Purified dendritic cells (1.74×10⁶ total) were incubated for 1 hour at 37° C. in a CO₂ incubator in EMEM containing 10% mouse serum with 1 mM of a cell surface peptide with 100% homology between human and mouse (Peptide #2) and having the following sequence

| | |
|---|---|
| CYYITGNLETFPRDPEK | SEQ. ID. No. 1 |

After washing the cells, CpG oligonucleotides (Qiagen) was added as adjuvant and the mixture was injected intradermally into one Balb/c mouse. Pre-immune serum had previously been obtained before the injection. Another bleed was obtained after 7 days and tested using ELISA.

### EXAMPLE 2

Mouse dendritic cells were generated in vitro by isolating monocytes from bone marrow and maturing them in the presence of IL-4, GM-CSF, and TNF-alpha to dendritic cells in 7 days. Peptide #2 was added at day 5. After washing the cells, CpG oligonucleotides (Qiagen) was added as adjuvant and the mixture was injected intradermally into one Balb/c mouse. Serum was obtained after 7 days and tested using ELISA.

### EXAMPLE 3

Antibodies in serum obtained from the immunized mouse of Example 1 were detected by ELISA. Two peptides, Peptide #2 and a control peptide having the following sequence:

| | |
|---|---|
| CTKTPLDQHTLQGDQA | SEQ. ID. No. 2 |

(referred to herein as “Peptide #1”), were dissolved in PBS followed by dilution to a final concentration of 4 microgram/ml in 0.1 sodium carbonate buffer having a pH of 8.6. Each well was coated with 25 microliters of peptide solution (100 ng per well) and the plate was incubated at 4° C. overnight. The wells were washed 3 times with nanopure water and subsequently blocked with 1% BSA in PBS for 1 hour at 37° C. Dilutions of serum, both preimmune and immune, were performed in 1% BSA/PBS. The primary incubation was for 1.5 hours at 37° C. followed by incubation with alkaline phosphatase-conjugated anti-mouse whole IgG (1:1000) dilution in 1% BSA/PBS.

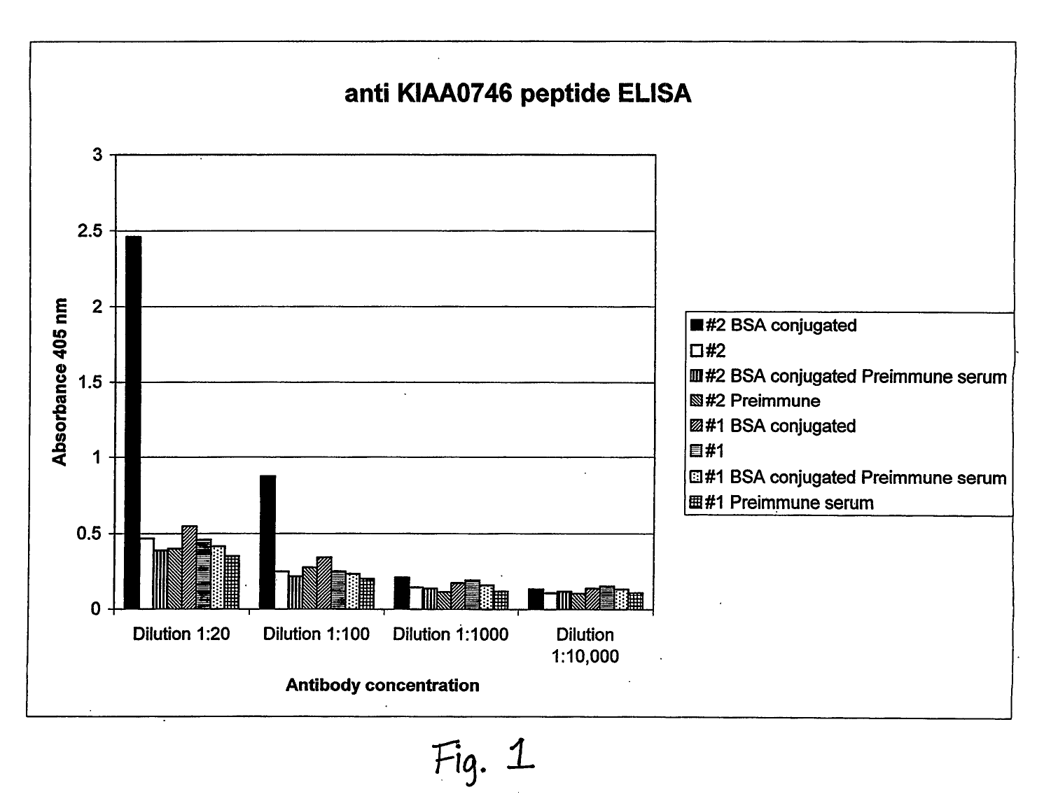

Absorbance was measured at 405 nm to determine the concentration of antibodies. For each peptide, Peptides #1 and #2, measurements were made of the peptide alone, preimmune serum containing the peptide, preimmune serum containing conjugated peptide, and serum containing conjugated peptide, at dilutions of 1:20, 1:100, 1:1,000 and 1:10,000. The results of this experiment are set forth in FIG. 1. As can be seen in FIG. 1, the mouse immunized with peptide loaded splenic dendritic cells developed antibodies against Peptide #2, whereas the irrelevant peptide, Peptide #1, was not recognized.

### EXAMPLE 4

Dendritic cells from mouse spleen were isolated by first digesting the splee 1 mg/ml collagenase for 1 hour, teasing the spleen apart and filtering the cells through a mesh 40 filter. Dendritic cells were positively selected using Stemcell Technologies' EasySep system labeling the cells with anti-CD11c coated magnetic beads. Purified dendritic cells (1.5×10⁶ total) were incubated for 1 hour at 37° C. in a CO₂ incubator in EMEM containing 10% mouse serum with 10 μg mSIGNR1, the mouse homologue of human L-SIGN (CD209L).

CpG oligonucleotides (Qiagen) were added as adjuvant and the mixture was injected subcutaneously into two Balb/c mice. Pre-immune serum had previously been obtained before the injection. Another bleed was obtained after 7 days and tested using ELISA.

### EXAMPLE 5

Antibodies in serum obtained from the immunized mouse of Example 4 were detected by ELISA. Anti-human Fc antibodies were coated at 4 μg/ml in PBS on ELISA plates and incubated overnight at 4° C. The next day, plates were blocked for 1 hour with 1% BSA in PBS, followed by 3 PBS washes. Recombinant mSIGNR1Fc fusion protein was added at 500 ng/ml and incubated on the plates for 2h at 37° C. Control plates were incubated with 1% BSA in PBS without mSIGNR1. After 3 PBS washes, serum obtained before and after immunization was added at 1:20 in PBS containing 1% BSA. Bound anti-mSIGNR1 antibody was detected with alkaline phosphatase-conjugated anti-mouse IgG followed by SigmaS substrate addition.

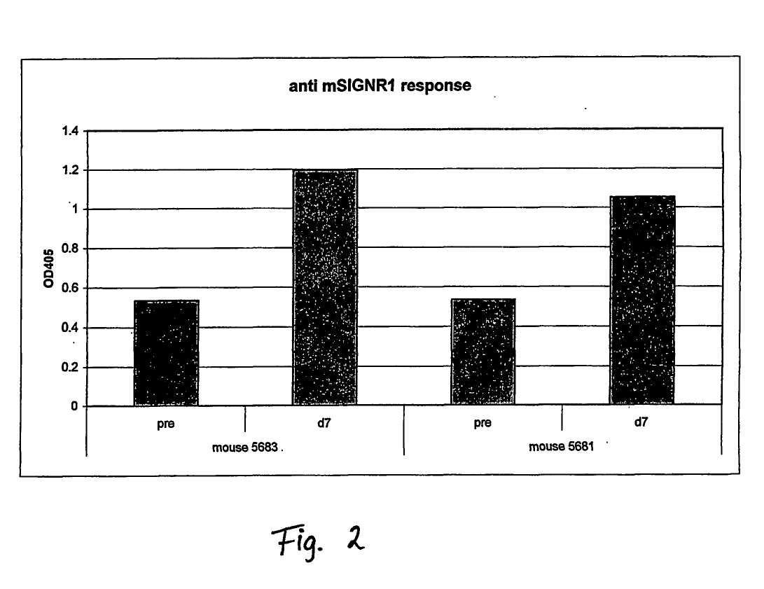

Absorbance was measured at 405 nm to determine the concentration of antibodies. The results of this experiment are set forth in FIG. 2. The OD on the BSA control wells was below 0.1 in all cases.

While the above description contains many specific details of methods in accordance with this invention, these specific details should not be construed as limitations on the scope of the invention, but merely as exemplifications of preferred embodiments thereof. Those skilled in the art will envision many other possible variations that all within the scope and spirit of the invention as defined by the claims appended hereto.

What is claimed is:

1. A method of producing an antibody comprising the steps of:
a) isolating dendritic cells from a host animal;
b) incubating the dendritic cells with a self peptide of interest to form an immunogen;
c) immunizing the host animal with the immunogen; and
d) harvesting cells that produce antibodies to the self peptide of interest.

2. A method as in claim 1 wherein the step of isolating dendritic cells from a host animal comprises utilizing a rodent as the host animal.

3. A method as in claim 1 wherein the step of isolating dendritic cells from a host animal comprises utilizing a mouse as the host animal.

4. A method as in claim 1 wherein the step of isolating dendritic cells comprises selecting dendritic cells utilizing antibodies to markers associated with dendritic cells selected from the group consisting of cell surface molecules responsible for T cell activation, adhesion molecules and costimulatory molecules.

5. A method as in claim 4 wherein the step of isolating dendritic cells comprises selecting dendritic cells utilizing antibodies to markers for dendritic cells selected from the group consisting of CD54 and CD11.

6. A method as in claim 1 wherein the step of incubating the dendritic cells with a self peptide of interest comprises selecting a self peptide that is homologous to a human peptide.

7. A method as in claim 1 wherein the step of incubating the dendritic cells with a self peptide of interest comprises incubating the dendritic cells and self peptide of interest for a period of time ranging from about 30 to about 180 minutes.

8. A method as in claim 1 wherein the step of incubating the dendritic cells with a self peptide of interest comprises incubating the dendritic cells and self peptide of interest that is coated onto a polystyrene bead.

9. A method as in claim 1 wherein the step of immunizing the host animal with the immunogen further comprises the step of adding an adjuvant to the immunogen.

10. A method as in claim 9 wherein the step of adding an adjuvant comprises selecting an adjuvant from the group consisting of IL-1, IL-2, IL-4, IL-5, IL-6, IL-7, IL-10, IL-12, IL-15, granulocyte-macrophage colony stimulating factor, granulocyte colony stimulating factor, interferon-γ, TNF-α, TGF-P, Flt-3, and CD40 ligand.

11. A method as in claim 9 wherein the step of adding an adjuvant comprises utilizing a CpG oligonucleotide as the adjuvant.

12. A method as in claim 1 wherein the step of immunizing the host animal with the immunogen further comprises the step of adding a B cells that have been previously incubated with the self peptide of interest to the immunogen.

13. A method as in claim 1 wherein said step of harvesting cells comprises removing cells comprising antibodies from the host, isolating RNA from the removed cells, constructing an antibody library and identifying one or more antibodies that bind to the self peptide of interest.

14. An antibody produced in accordance with the method of claim 1.

15. A method of producing an antibody comprising the steps of:
a) isolating dendritic cells from a host animal;
b) transfecting the dendritic cells with nucleic acid encoding a self peptide of interest to form an immunogen;
c) immunizing the host animal with the immunogen; and
d) harvesting cells that produce antibodies to the self peptide of interest.

16. A method of producing a human antibody comprising the steps of:
a) grafting human tissue into an immunodeficient, non-human, mammalian host animal, the human tissue comprising at least a sufficient portion of thymus, spleen, bone marrow, skin, lymph nodes and fetal liver to produce a human antibody;
b) applying a composition to the grafted human tissue, the composition including at least one growth factor and a material to support cell growth;
c) obtaining human monocytes from a donor;
d) maturing the human monocytes to dendritic cells;
e) isolating the dendritic cells;
f) incubating the dendritic cells with a self peptide of interest to form an immunogen;
g) immunizing the host with the immunogen; and
h) harvesting at least a portion of the grafted tissue that includes cells that produce human antibodies.

17. A method as in claim 16 where the immunodeficient, non-human, mammalian host is a rodent.

18. A method as in claim 16 where the immunodeficient, non-human, mammalian host is a SCID mouse.

19. A method as in claim 16 further comprising the step of irradiating the immunodeficient, non-human, mammalian host prior to said grafting step.

20. A method as in claim 16 further comprising the step of testing for the presence of human antibodies prior to said immunizing step.

21. A method as in claim 16 wherein the step of obtaining human monocytes comprises obtaining monocytes from a donor selected from the group consisting of a donor of the human tissue and an HLA-matched human donor.

22. A method as in claim 16 wherein said step of harvesting cells comprises removing at least a portion of the human tissue from the host, isolating RNA from the removed human tissue, constructing an antibody library and identifying one or more antibodies that bind to the self peptide of interest.

23. A human antibody produced in accordance with the method of claim 16.

24. A method of producing a human antibody comprising the steps of:
a) grafting human tissue into an immunodeficient, non-human, mammalian host animal, the human tissue comprising at least a sufficient portion of thymus, spleen, bone marrow, skin, lymph nodes and fetal liver to produce a human antibody;
b) applying a composition to the grafted human tissue, the composition including at least one growth factor and a material to support cell growth;
c) linking a self peptide of interest to an antibody to DC-Specific ICAM-3 grabbing non-integrin;
d) immunizing the host with the self peptide of interest and antibody to DC-Specific ICAM-3 grabbing non-integrin; and
e) harvesting at least a portion of the grafted tissue that includes cells that produce human antibodies to the self peptide of interest.

25. A human antibody produced in accordance with the method of claim 24.

26. A method of testing the effect of drugs comprising:
administering a drug to a non-human mammalian having at least a sufficient portion of human thymus, spleen, bone marrow, skin, lymph nodes and fetal liver grafted therein to allow the development of one or more types of human immune cells selected from the group consisting of T cells, B cells and dendritic cells; and
observing any effects of the drug on human antibody production.

27. A method of producing a human antibody comprising the steps of:
a) injecting CD34+ human cord blood into RAG/γc −/− mice to produce a human antibody;
b) maturing the cells from CD34+ human cord blood to dendritic cells;
c) isolating the dendritic cells;
d) incubating the dendritic cells with a self peptide of interest to form an immunogen;
e) immunizing the injected RAG/γc −/− mice with the immunogen; and
f) recovering human antibodies.
